(19) United States
(12) Patent Application Publication
(10) Pub. No.: US 2012/0004616 A1
(43) Pub. Date: Jan. 5, 2012

(54) GASTRIC PORT SYSTEM

(76) Inventor: Abhishek Mitra, Winston Salem, NC (US)

(21) Appl. No.: 12/976,537

(22) Filed: Dec. 22, 2010

Related U.S. Application Data
(60) Provisional application No. 61/291,214, filed on Dec. 30, 2009.

(51) Int. Cl. A61M 39/00

(52) U.S. Cl. 604/175

(57) ABSTRACT

A gastric port system for transport of materials to the interior of a body cavity that may include a tube configured to be disposed between a first body cavity and an area adjacent to the first body cavity. The gastric port system may further include a first bolster that includes a curved component having an open channel defined on a side of the curved component.

This application claims priority to U.S. Provisional Application Ser. No. 61/291,214, filed Dec. 30, 2009.

### BACKGROUND

The present system relates to medical devices, and more particularly, to low-profile external fittings for systems that deliver materials to the interior of a body cavity.

Patients for which normal ingestion of food becomes difficult or impossible may require placement of a feeding tube to assist in providing their nutritional needs. For some individuals, such as comatose patients, stroke victims, or those with a compromised gastrointestinal tract and the like, this may require placement of a tube that is introduced percutaneously into the stomach for delivery of nutritional products directly into the stomach or jejunum. The procedure, known as a Percutaneous Endoscopic Gastrostomy (PEG) can be performed using several different techniques. Some techniques include the introduction of an endoscope into the stomach. The desired site where the stoma is to be created is indicated from above by depressing the abdomen and viewing the depressed site with the endoscope. Transillumination may also be utilized to locate the desired site through the abdominal wall. A sheathed needle or trocar punctures the abdominal wall and enters the stomach, creating a stoma. The needle is removed and a looped insertion wire/suture is introduced through the sheath where it is grasped by a snare or forceps deployed from the working channel of the endoscope. Once it is captured, the insertion wire/suture is pulled into the working channel of the endoscope. The endoscope is then withdrawn from the patient via the oral cavity, pulling the insertion wire/suture with it.

In the standard Ponsky method (or “pull” method), the distal loop of a percutaneous gastrostomy feeding tube is coupled to the insertion wire/suture exiting the patient's mouth. With the insertion wire/suture now tethered to the gastrostomy feeding tube, the endoscopist retracts the portion of the insertion wire/suture exiting the stoma, thereby pulling the gastrostomy feeding tube into the patient's mouth and towards the stomach. With continued retraction of the insertion wire/suture, the distal end of the gastrostomy feeding tube is pulled out through the stoma. The gastrostomy feeding tube typically includes a tapered dilator portion to aid its passage through the stoma. Once the feeding tube has been properly positioned with the proximal end cap or bolster of the feeding tube against the internal wall of the stomach, it is secured by an external bolster positioned against the outside of the abdomen wall.

In a variation of the PEG procedure known as the “push” method, the gastrostomy feeding tube is advanced or pushed down the esophagus by the physician and into position in the stomach using a wire guide that has been placed in the same manner as the insertion wire in the “pull” method. More specifically, the feeding tube is loaded on the portion of the wire guide exiting the patient's mouth by passing the end of the wire guide through a lumen extending through the length of the feeding tube. While holding the wire guide stationary, the physician pushes the feeding tube along the wire guide through the patient's mouth, into the stomach, and then out through the stoma. The feeding tube is then secured in the same manner as the “pull” method.

Yet another method is simply to insert the feeding tube through the patient's abdominal wall using the Seldinger technique and bypass insertion through the mouth. However, this method typically requires the deployment of an internal retention device including, and/or in addition to attaching a bolster to the interior portion of the feeding tube, which may need to be delivered and attached endoscopically.

As stated above, typically, a retention bolster is positioned against the inside and/or outside of the abdomen wall, or whichever body cavity or area the gastric port is being used in relation to. The bolster is present to keep in place and support the gastric port system and prevent sudden or unexpected removal of the tube from the stoma site. Bolsters for supporting tubes, such as feeding tubes, inside or outside the body have generally focused on maintaining the secure anchoring of the device to the patient. To provide support, bolsters have employed flanges, cross-bars, discs, or balloons for contacting the surface of the tissue. In the past, however, bolsters have tended to increase the localized pressure at the exit site, especially when the port, either accidentally or intentionally, is moved thereabout.

For both ambulatory and bed-ridden patients, an external length of feeding tube and/or feeding apparatus may be connected to and removed from an external fitting of the tube, with or without an external bolster. Different diameters and lengths of tube may be needed. Depending on application, the system may need to be present with the patient for an extended period of time. Many current bolsters have a large and/or awkward profile such that extended use makes them uncomfortable for the patient where normal bodily movement is necessary. Further, many external bolsters have the disadvantage of having to be threaded over the tube.

A need therefore exists for a bolster that has a lower profile that is configured for use adjacent to a body surface that can decrease the localized pressure at the exit site, especially when the tube and/or bolster, either accidentally or intentionally, is moved thereabout.

### BRIEF SUMMARY

The foregoing problems are solved and technical advance is achieved with an illustrative gastric port system. The system has the advantage of being able to laterally couple the external bolster to the tube.

These and other advantages, as well as the gastric port system itself, will become apparent in the details of construction and operation as more fully described below. Moreover, it should be appreciated that several aspects of the invention can be used with other types of gastric port systems or medical devices.

### BRIEF DESCRIPTION OF THE DRAWINGS

FIG. 1 is an illustration of a gastric port system.

FIG. 2 is a perspective view of the low profile external bolster illustrated in FIG. 1.

FIG. 3 is a side view of the low profile external bolster illustrated in FIG. 1.

FIG. 4 is a top view of the low profile external bolster illustrated in FIG. 1.

### DETAILED DESCRIPTION OF THE DRAWINGS AND THE PRESENTLY PREFERRED EMBODIMENTS

For the purposes of promoting an understanding of the principles of the gastric port system, reference will now be made to the embodiments illustrated herein. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

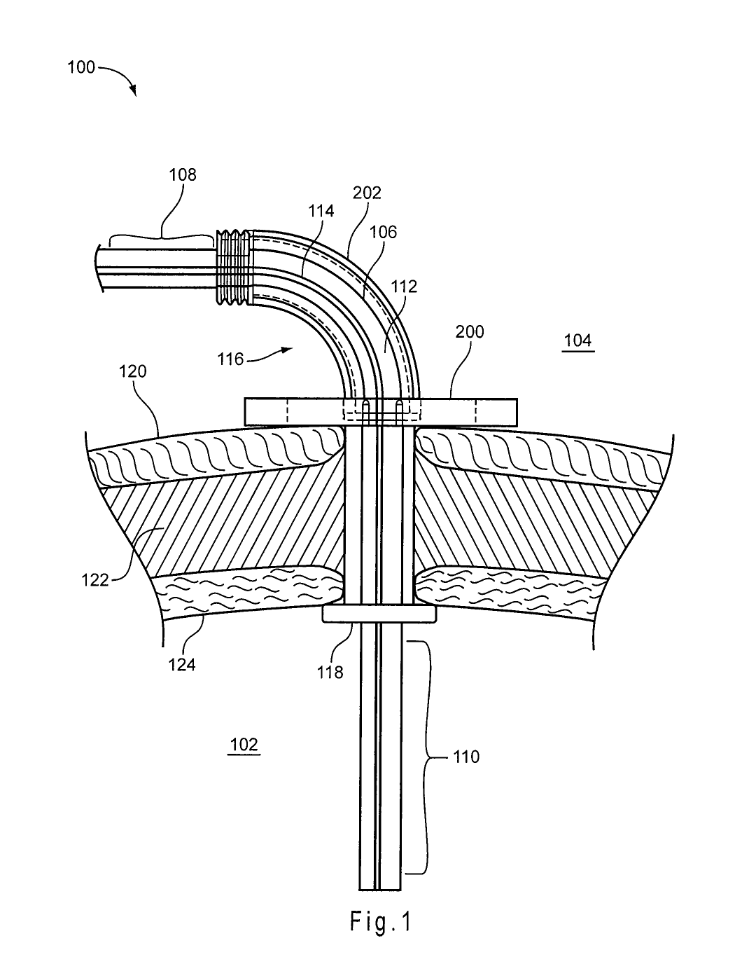

Referring now to FIG. 1, a gastric port system 100 is shown transversing a stoma between a first body cavity 102 and an area 104 adjacent to the first body cavity 102. More specifically, and as illustrated in FIG. 1, the first body cavity 102 may be the abdominal cavity of the patient and area 104 illustrated is an area external to the patient. However, the first body cavity 102 may be any body cavity that would benefit from the advantages disclosed herein.

Gastric port system 100 includes a tube or port 106 that has a proximal portion 108 and a distal portion 110. The proximal portion 108 may be defined as the portion of the tube 106 that extends into area 104 and the distal portion 110 may be defined as the portion of the tube 106 that extends into the first body cavity 102. The tube 106 also includes a passageway 112 that allows for the passage of materials through the tube 106. More specifically, the passageway 112 allows for the passage of nutritional products or medications directly into the body or body cavity of a patient. Alternatively, a catheter 114 may be introduced to extend through the passageway 112 and may act as the delivery mechanism for materials directly into the body or body cavity of a patient. The tube 106 may be made of any material suitable for the placement of the tube 106 into a body or body cavity of a patient. Likewise, the catheter 114 may be made of any material suitable for delivery of, for example, nutritional products or medications into the body or body cavity.

A first bolster 116 and a second bolster 118 are shown in FIG. 1. The first bolster 116 may be slidable relative to the tube 106. The second bolster 118 may be fixably attached to the distal portion 110 of the tube 106. FIG. 1 shows the distal portion 110 of the tube 106 extending completely through the bolster 118; however, it need not do so and may instead terminate at or in the second bolster 118. The first bolster 116 may be slidably disposed on the proximal portion 108 of the tube 106. Alternatively, the bolster 116 may be placed about the tube 106, as further described below. The first bolster 116 and the second bolster 118 may be different sizes and shapes. For example, the bolster 116 may be molded or machined from a solid piece of material, such as medical grade silicone and the like and may be ring shaped, bowl shaped, T-shaped, Malecot shaped, mushroom shaped, dome shaped, conical shaped, or any other shape that can provide retention for the tube 106. Alternatively, the bolster 116 may be formed such that the components thereof are formed as unitary structures and then assembled. Similarly, the bolster 118 may be molded or machined from a solid piece of material, such as medical grade silicone and the like and may be ring shaped, bowl shaped, T-shaped, Malecot shaped, mushroom shaped, dome shaped, conical shaped, or any other shape that can provide retention for the port 106.

FIG. 1 illustrates the first bolster 116 engaging the first side 120 of a bodily wall 122. The bodily wall 122 may be the material or space separating the body cavity 102 and area 104. The second bolster 118 is shown engaging a second side 124 of the bodily wall 122.

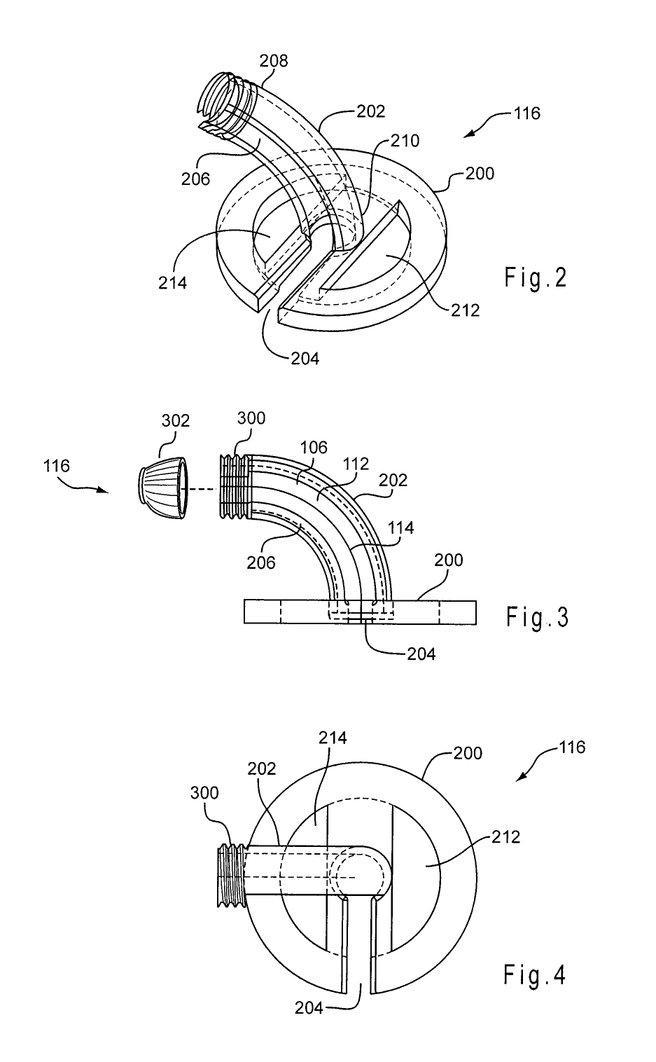

Referring now to FIGS. 2-4, the bolster 116 of FIG. 1 is shown. Bolster 116 includes a base 200 and a curved component 202 to which the base 200 is attached. Preferably, the bolster 116 is low profile relative to the first side 120 of the bodily wall 122. A low-profile design of the bolster 116 allows for the bolster 116 to minimize being caught on clothing and/or noticeable by others under the patient's clothing. To accomplish the low-profile design, the curved component 202 may be elbow-shaped, bent, bowed, arched, or any similar shape that promotes conforming of the tube 106 to the curved component 202 when the tube 106 is laterally placed through the open channel 206 and the open channel 204 of the bolster 116, without kinking the tube. A proximal end 208 of the curved component 202 is substantially parallel to the first side of the bodily wall 120 (not shown) and a distal end 210 of the curved component 202 is substantially perpendicular to the first side of the bodily wall 120 (not shown). The base 200 has an open channel 204 that is large enough to receive the tube 106, which may be pushed laterally through the open channel 204. Preferably, the width of the open channel 204 of the base 200 is narrower than an outside diameter of the tube 106, and the tube 106 is sufficiently flexible to pass through the open channel 204 of the base 200. The open channel 204 may be aligned with an open channel 206 defined on a side of the curved component 202 which extends along a length of the curved component 202. Preferably, the open channel 204 is in communication with the open channel 206 such that the channels 204 and 206 may be configured for the passage of the tube 106 laterally therethrough. The base 200 preferably has two openings 212 and 214 that are located near the distal end 210 of the curved component 202. Openings 212 and 214 serve to allow air flow and/or air to the bodily wall when the bolster 116 is in use.

Referring now specifically to FIG. 3, the curved component 202 may have a threaded end 300 onto which a threaded cap 302 or securing mechanism may be threadably engaged and configured to prevent the tube from being removed from the open channel 206 of the curved component 202. The cap 302 may be threaded onto the threaded end 300 by, for example, screwing the cap 302 onto the threaded end 300 after the tube 106 is laterally placed through the base 200 and curved component 202. Screwing the cap 302 onto the threaded end 300 acts to secure the tube 106 by crimping the curved component 202 against the end of the tube 106 when engaged with the curved component 202 so as to prevent axial movement or lateral movement or both of the tube 106 relative to the curved component 202. The threaded end 300 and cap 302 allows the bolster 116 to receive varying tube 106 sizes, and is therefore appropriately sized to receive varying tube 106 sizes. Securing mechanisms other than the cap 302 may also be used, such as a collar, a band, and the like such that the securing mechanism may prevent axial movement or lateral movement or both of the tube 106 within the bolster 116.

Referring to FIGS. 1-4, after a stoma is created, the tube 106 may be inserted into the stoma according methods described above. Unlike prior bolsters, however, the bolster 116 need not be fed over the proximal end 208 of the port 106. Instead, the tube 106 may be positioned by sliding or placing the tube 106 laterally through the open channel 204 and open channel 206 of the bolster 116. The shape of the curved component 202 is configured to allow passage of the tube 106 when it is laterally placed through the open channel 206 and the open channel 204 of the bolster 116. When so placed, the tube 106 will gently (i.e., preferably without kinking or crimping) curve toward the proximal end 208 of the bolster 116, fitting snugly within the open channel 206. Once the tube 106 is positioned, the cap 302 may be screwed onto the threaded end 300 of the bolster 116, thereby securing the tube 106 within the bolster 116.

It is therefore intended that the foregoing detailed description be regarded as illustrative rather than limiting, and that it be understood that it is the following claims, including all equivalents, that are intended to define the spirit and scope of this invention.

1. A gastric port system for transport of materials to the interior of a body cavity comprising:
a tube configured to be disposed between a first body cavity and an area adjacent to the first body cavity and extending through a bodily wall separating the adjacent area from the first body cavity; the tube further comprising a distal portion, a proximal portion, and a passageway therethrough;
a first bolster configured to engage a first side of the bodily wall, the bolster including a curved component having an open channel defined on a side of the curved component and a base fixably attached to the curved component, the base having an open channel defined on a side of the base in communication with the open channel of the curved component, the channels each being configured for the passage of the tube laterally therethrough; and
a second bolster configured to engage a second side of the bodily wall disposed on the distal portion of the tube.

2. The gastric port system of claim 1 further comprising a catheter for the passage of materials, the catheter being removably disposed in the passageway of the tube and extending through at least a portion of the tube.

3. The gastric port system of claim 1 further including a securing mechanism removably engaged with the curved component and configured to prevent the tube from being removed from the passageway of the curved component.

4. The gastric port system of claim 3, wherein the securing mechanism comprises a threaded portion that engages with a threaded end of the curved component.

5. The gastric port system of claim 3, wherein the securing mechanism is configured to crimp the curved portion against the tube when engaged with the curved portion so as to prevent axial movement of the tube relative to the curved component.

6. The gastric port system of claim 1 further including a securing mechanism adapted to attach to the first bolster to prevent at least one of lateral and axial movement of the tube relative to the curved component of the bolster.

7. The gastric port system of claim 1 where a width of the open channel of the base is narrower than an outside diameter of the tube, and the tube is sufficiently flexible to pass through the open channel of the base.

8. The gastric port system of claim 1 where the base has a first opening that allows air flow through the base.

9. The gastric port system of claim 1 where the base has a first opening and a second opening that allows air flow through the base.

10. A low-profile external bolster comprising:
a curved component having a proximal end and a distal end, the curved component further including a first open channel defined on a side of the curved component that is configured to receive a tube laterally therethrough; and
a base fixably attached to the curved component, the base including a second open channel defined on a side of the base that is aligned with the first open channel of the curved component and is configured to receive the tube laterally therethrough.

11. The low-profile external bolster of claim 10, where a securing mechanism is threadably engaged with the proximal end of the curved component.

12. The low-profile external bolster of claim 11 where the securing mechanism is a cap.

13. The low-profile external bolster of claim 10 further including a securing mechanism adapted to attach to the curved component to prevent axial movement of the tube within the bolster.

14. The low-profile external bolster of claim 10, where the base further includes a first opening that allows airflow through the base.

15. The low-profile external bolster of claim 10, where the base further includes a first opening and a second opening that allows airflow through the base.

16. The low-profile external bolster of claim 10, where a width of the open channel of the base is narrower than an outside diameter of the tube, and the tube is sufficiently flexible to pass through the open channel of the base.

17. A low-profile external bolster comprising:
a curved component configured to receive a tube, having a proximal end and a distal end, the curved component further including a first open channel defined on a side of the curved component;
a securing mechanism adapted to attach to the curved component to prevent at least one of lateral and axial movement of a tube within the bolster; and
a base fixably attached to the curved component, the base including a second open channel aligned with the first open channel of the curved component, where the second open channel and the first open channel are configured to receive a tube.

18. The low-profile external bolster of claim 17 where the base further includes a first opening and a second opening that allows airflow through the base.

19. The low-profile external bolster of claim 17 where the securing mechanism is a threaded cap.

20. The low-profile external bolster of claim 17, where a width of the open channel of the base is narrower than an outside diameter of the tube, and the tube is sufficiently flexible to pass through the open channel of the base.
